(19) United States
(12) Patent Application Publication
(10) Pub. No.: US 2026/0348897 A1
(43) Pub. Date: Nov. 5, 2026

(54) GAS SENSOR MODULE AND METHOD FOR MANUFACTURING GAS SENSOR MODULE

(71) Applicant: NISSHA CO., LTD., Kyoto-shi, Kyoto (JP)

(72) Inventor: Hitoshi KATAYAMA, Sanda-shi, Hyogo (JP)

(21) Appl. No.: 19/488,542

(22) PCT Filed: May 10, 2024
(86) PCT No.: PCT/JP2024/017529

§ 371 Date: Nov. 26, 2025

(30) Foreign Application Priority Data
May 29, 2023 (JP) 2023-087505

(51) Int. Cl. G01N 27/12

(52) U.S. Cl. CPC G01N 27/122

(57) ABSTRACT

A gas sensor module includes a gas sensor, a substrate, a waterproof molded body and container. The gas sensor is configured to detect gas heavier than air. The substrate includes an electric circuit connected to the gas sensor. The waterproof molded body covers a portion of the substrate including the electric circuit and a portion of the gas sensor to waterproof the covered portions. The container houses the gas sensor and the substrate in an internal space. The container includes a gas inlet opened downward to introduce the gas heavier than air into the internal space, and a drain port that functions as an air vent hole for introducing the gas and drains water entering the internal space. The substrate is fixed to the container so that an opening of the gas sensor faces the gas inlet.

### CROSS REFERENCES TO RELATED APPLICATIONS

This application is a U.S. National stage of International Application No. PCT/JP2024/017529, filed on May 10, 2024. This application claims priority to Japanese Patent Application No. 2023-087505 filed on May 29, 2023 with Japan Patent Office.

### BACKGROUND

### Field of the Invention

The present invention relates to a gas sensor module and a method for manufacturing the gas sensor module, and relates to a gas sensor module including a gas sensor that detects gas heavier than air, and a method for manufacturing the gas sensor module.

### Background Information

A gas sensor may be required to sense gas in an environment where water may enter. In such an environment, a gas sensor module may be provided with waterproof property to prevent entry of water. For example, as described in Patent Literature 1 (JP 5166376 B), a gas sensor is housed inside a case (container) to improve waterproof performance.

### SUMMARY

A gas sensor needs to introduce gas to be detected into the sensor in order to detect the gas to be detected. Thus, the gas sensor cannot be housed in a container and completely sealed. For example, in a case where gas is detected in equipment installed outdoors, transportation equipment such as automobiles, and human bodies, rainwater, water in water areas such as seawater, and water derived from animals and plants such as sweat and fruit juice may enter the container.

Thus, even if the gas sensor is housed in the container, in an environment where water enters the container, it is also necessary to provide waterproof property to the gas sensor housed in the container and an electric circuit connected to the gas sensor.

One object is to improve waterproof property of a gas sensor housed in a container and an electric circuit to which the gas sensor is connected.

Hereinafter, a plurality of aspects will be described as means for solving the problem. These aspects can be combined in any manner if necessary.

A gas sensor module according to one aspect of the present disclosure includes a gas sensor that detects gas heavier than air, a substrate including an electric circuit connected to the gas sensor, a waterproof molded body that covers a portion of the substrate including the electric circuit and a portion of the gas sensor to waterproof the covered portions, and a container that houses the gas sensor and the substrate in an internal space. The container includes a gas inlet opened downward to introduce the gas heavier than air into the internal space, and a drain port that functions as an air vent hole for introducing the gas and drains water entering the internal space. The substrate is fixed to the container so that an opening of the gas sensor faces the gas inlet. According to the gas sensor module according to the first aspect, it is possible to prevent water from being splashed on the gas sensor and the substrate while allowing the gas outside the container to be guided to the gas sensor by the container. Furthermore, the waterproof molded body can prevent water from entering the electric circuit.

The gas sensor module described above can be configured so that the container has a lower wall provided with the gas inlet, and supports the substrate with a gap of 0.5 mm or more between a lower end of the gas sensor and the lower wall. In the gas sensor module configured as described above, water droplets generated on the lower wall of the container hardly enter the gas sensor.

The gas sensor module described above can be configured so that the container has a rib protruding into the internal space, the waterproof molded body is a hot melt mold, the substrate has higher rigidity than the waterproof molded body, and a portion of the substrate exposed from the waterproof molded body is supported and fixed by the rib. The gas sensor module configured as described above is supported by the rib, so that it is possible to reduce the number of places where the gas sensor, the substrate, and the waterproof molded body are in contact with an inner surface of the container, so that it is possible to suppress transfer of water from the container to the gas sensor, the substrate, and the waterproof molded body.

The gas sensor module described above can be configured so that the container includes a protrusion for attaching inspection equipment around the gas inlet. In the gas sensor module configured as described above, the protrusion facilitates attachment of the inspection equipment. In addition, it is possible to reduce water droplets entering from the gas inlet by the protrusion.

In the gas sensor module described above, the container includes, as the drain port, a first elongated hole and a second elongated hole provided on both sides of the gas inlet. The gas sensor module configured as described above can have high drainage capacity and high functionality as the air vent hole.

The gas sensor module described above further includes a cable connected to the electric circuit, the container has a cable outlet for taking out the cable from the substrate to the outside of the container, and the waterproof molded body covers a portion of the cable and fills a gap between the cable outlet and the cable to seal the cable outlet. The gas sensor module configured as described above can prevent water from entering the internal space of the container along the cable.

A method for manufacturing a gas sensor module according to one aspect of the present disclosure includes a first step and a second step. In the first step, a waterproof molded body is formed by hot melt molding so that a portion of a gas sensor that detects gas heavier than air and a portion of a substrate including an electric circuit in the substrate including the electric circuit connected to the gas sensor are covered with the waterproof molded body. In the second step, the substrate is made to abut on the container and fixed to the container so that an opening of the gas sensor faces a gas inlet of the container opened downward to introduce gas heavier than air into an internal space of the container, and a gap is formed between a lower end of the gas sensor and a lower wall of the container. In the method for manufacturing the gas sensor module configured as described above, a portion of the gas sensor and a portion of the substrate can be quickly and accurately covered with the waterproof molded body by the hot melt molding.

According to the gas sensor module of the present disclosure, it is possible to improve waterproof property of the gas sensor housed in the container and the electric circuit to which the gas sensor is connected. According to the method for manufacturing the gas sensor module of the present disclosure, it is possible to easily manufacture the gas sensor module having improved waterproof property of the gas sensor housed in the container and the electric circuit to which the gas sensor is connected.

### BRIEF DESCRIPTION OF THE DRAWINGS

FIG. 1 is a front view illustrating a gas sensor module according to an embodiment.

FIG. 2 is a bottom view of the gas sensor module illustrated in FIG. 1.

FIG. 3 is a left side view of the gas sensor module illustrated in FIG. 1.

FIG. 4 is a right side view of the gas sensor module illustrated in FIG. 1.

FIG. 5 is a top view of the gas sensor module illustrated in FIG. 1.

FIG. 6 is a rear view of the gas sensor module illustrated in FIG. 1.

FIG. 7 is a front view illustrating a state where a lid portion is removed from the gas sensor module in FIG. 1.

FIG. 8 is a rear view illustrating the lid portion of the gas sensor module in FIG. 1.

FIG. 9 is a front view of an assembly of a gas sensor, a substrate, a waterproof molded body, and a cable according to the embodiment.

FIG. 10 is a rear view illustrating an assembly of the substrate, the waterproof molded body, and the cable in FIG. 9.

FIG. 11 is a cross-sectional view illustrating the gas sensor module in FIG. 1.

FIG. 12 is an enlarged cross-sectional view illustrating a periphery of a gas inlet illustrated in FIG. 11.

FIG. 13 is an enlarged cross-sectional view illustrating the periphery of the gas inlet of the gas sensor module in FIG. 1.

FIG. 14 is an enlarged cross-sectional view illustrating a periphery of a cable outlet of the gas sensor module in FIG. 1.

FIG. 15 is a cross-sectional view illustrating the gas sensor module in FIG. 1.

FIG. 16 is a rear view illustrating the gas sensor and the substrate according to the embodiment.

FIG. 17 is a front view illustrating a gas sensor module according to a modification.

FIG. 18 is a front view illustrating a state in which a lid portion is removed from the gas sensor module in FIG. 17.

FIG. 19 is a bottom view of the gas sensor module illustrated in FIG. 17.

FIG. 20 is a view for explaining accumulation of dust in a case where a bank portion is provided on an upper surface of a lower wall.

### DETAILED DESCRIPTION OF EMBODIMENTS

### (1) Overall Configuration

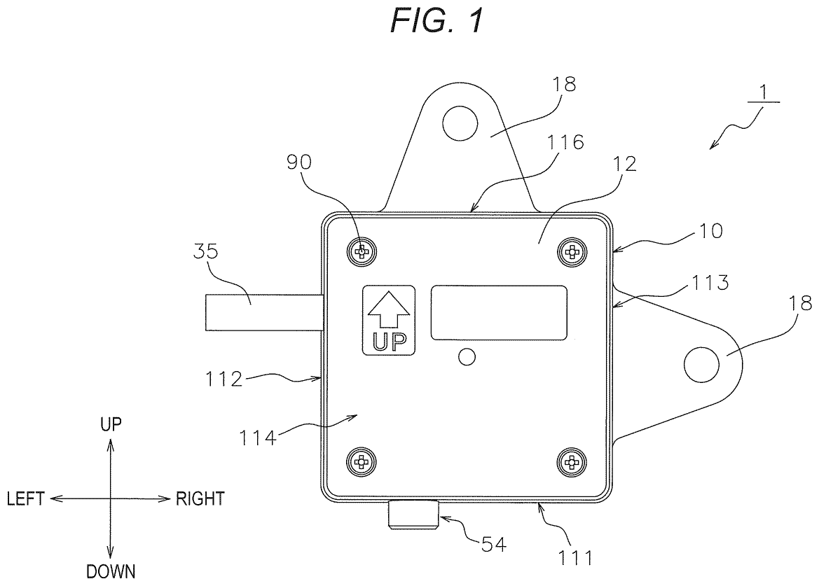

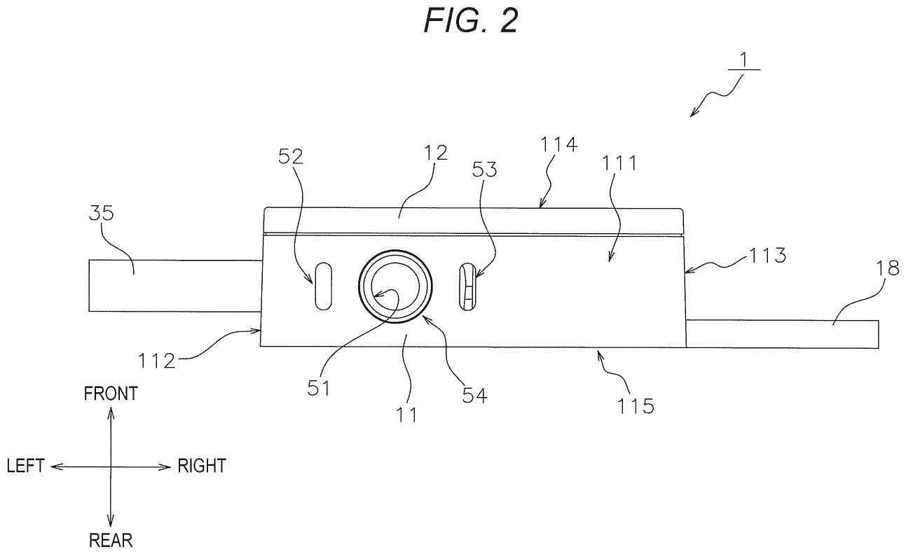

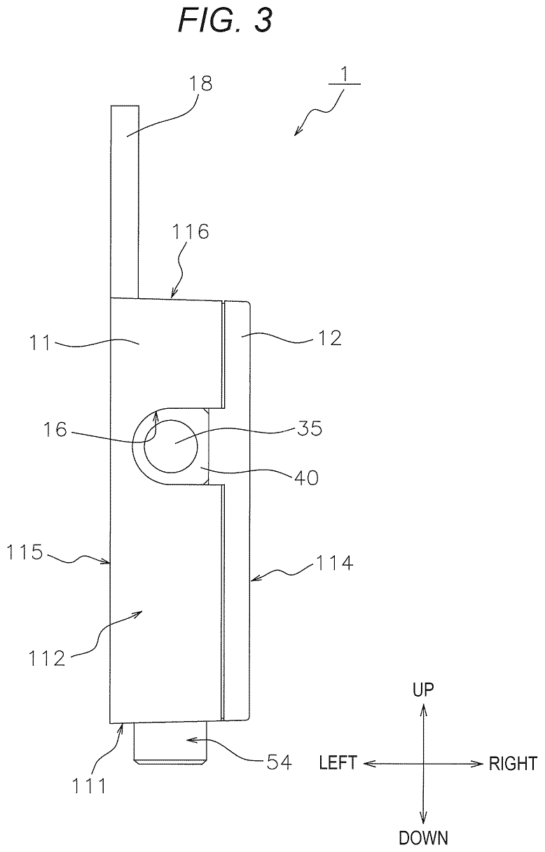

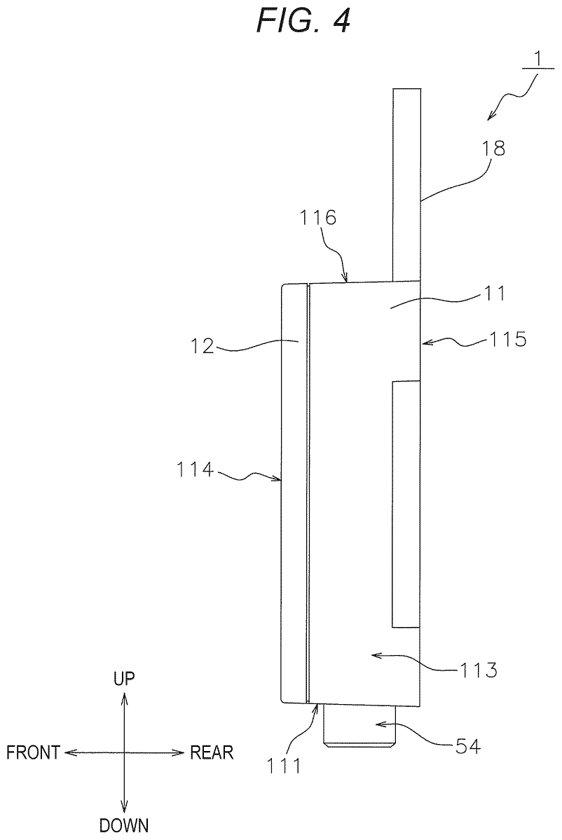

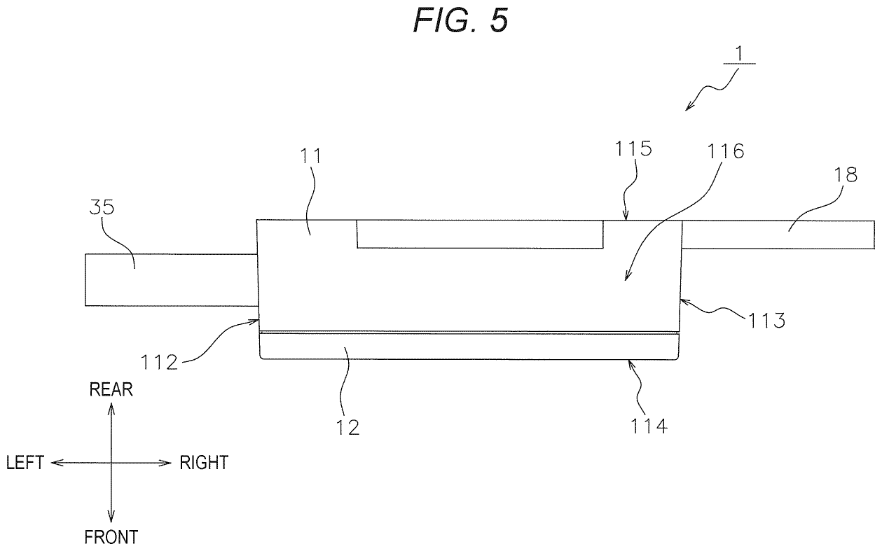

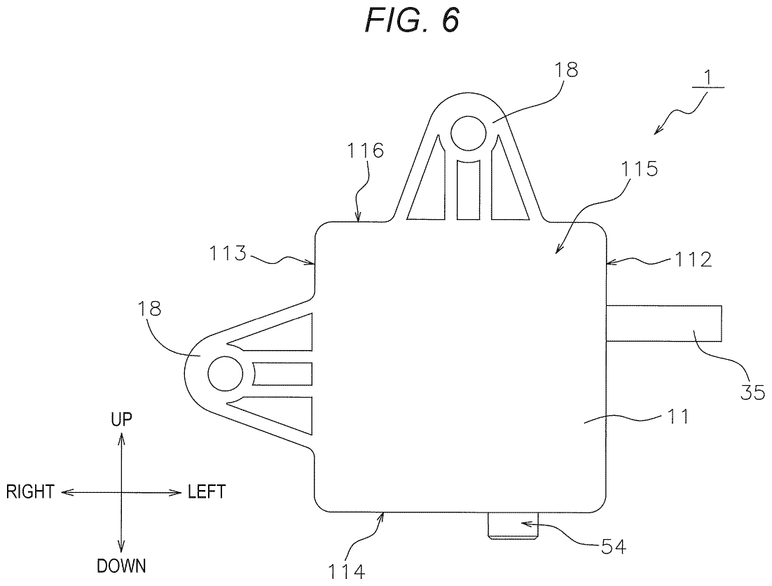

FIG. 1 illustrates appearance of a gas sensor module 1 as viewed from the front, FIG. 2 illustrates the appearance as viewed from the bottom, FIG. 3 illustrates the appearance as viewed from the left, FIG. 4 illustrates the appearance as viewed from the right, FIG. 5 illustrates the appearance as viewed from the top, and FIG. 6 illustrates the appearance as viewed from a rear side. In other words, FIG. 1 is a front view, FIG. 2 is a bottom view, FIG. 3 is a left side view, FIG. 4 is a right side view, FIG. 5 is a top view, and FIG. 6 is a rear view of the gas sensor module.

The container 10 includes a housing portion 11 and a lid portion 12. The appearance of the gas sensor module 1 is mainly the appearance of the container 10. The container 10 has a lower wall 111, a left side wall 112, a right side wall 113, a front wall 114, a rear wall 115, and an upper wall 116. A direction in which the container 10 is attached is determined, and the container 10 is attached so that a direction of “UP” indicated by an arrow drawn on the lid portion 12 faces upward. The “UP” and the arrow are drawn with irregularities formed on a surface of the container 10. As described above, the direction in which the gas sensor module 1 is attached is determined, and thus, the arrow indicating the direction of the gas sensor module 1 is illustrated in each drawing. In the description of the present specification, a lower side is a direction pulled by gravity. The container 10 is attached by inserting a screw into a hole of an attachment portion 18, for example, inside equipment that uses gas to be detected. However, the attachment method may be a method other than screwing, and for example, the attachment portion 18 may be attached to a wall, or the like, of the equipment with an adhesive.

The container 10 is formed with, for example, a resin, ceramic, or a composite material in which a filler is mixed in a resin. Examples of the resin include a thermoplastic resin. Examples of the thermoplastic resin include a polypropylene resin, an ABS resin, a polyethylene terephthalate resin, a polycarbonate resin, a PBT (polybutylene terephthalate) resin, a polyamide resin, a PE (polyethylene) resin, a POM (polyoxymethylene) resin, a Teflon (registered trademark) resin, and an acrylic resin. The container 10 is, for example, manufactured by injection molding, and in a case where the container 10 is manufactured by injection molding, a thermoplastic resin is preferably used.

The lid portion 12 forms the front wall 114. The front wall 114 has a shape based on a rectangle when viewed from the front. A shape of each of four corners of the front wall 114 is an arc when viewed from the front. Screws 90 for fastening the lid portion 12 are disposed at the four corners of the front wall 114.

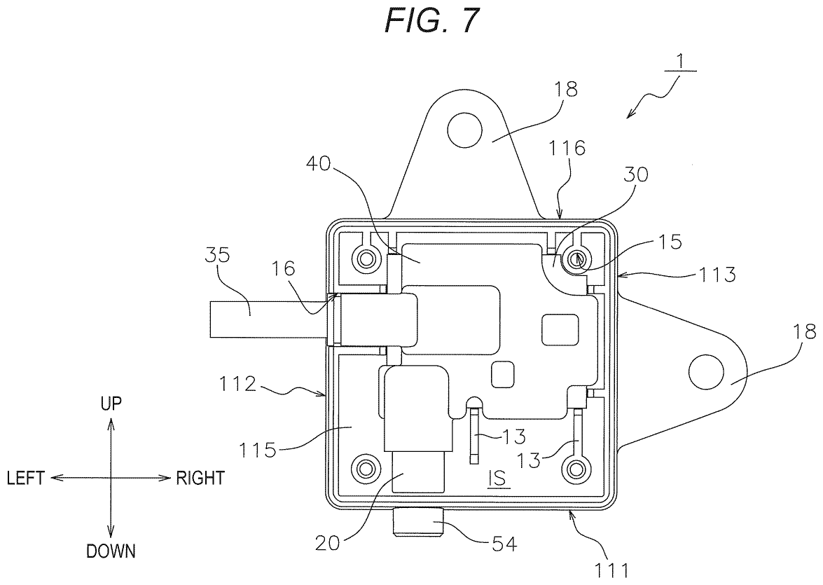

The housing portion 11 mainly forms the lower wall 111, the left side wall 112, the right side wall 113, the rear wall 115, and the upper wall 116. The container 10 has an internal space IS surrounded by the lower wall 111, the left side wall 112, the right side wall 113, the front wall 114, the rear wall 115, and the upper wall 116 (see FIG. 7). FIG. 7 is a front view of the gas sensor module 1 from which the lid portion 12 is removed as viewed from the front.

The gas sensor module 1 includes a gas sensor 20, a substrate 30, and a waterproof molded body 40 in addition to the container 10.

The gas sensor 20 is a device that detects gas heavier than air. In other words, density of the gas to be detected by the gas sensor 20 is greater than density of air. Examples of the gas to be detected include propane gas, carbon dioxide gas, nitrogen monoxide gas, argon gas, and chlorofluorocarbon gas heavier than air. Examples of the chlorofluorocarbon gas heavier than air include hydrofluorocarbon gas heavier than air. Examples of the hydrofluorocarbon gas heavier than air include tetrafluoropropene gas and difluoromethane gas.

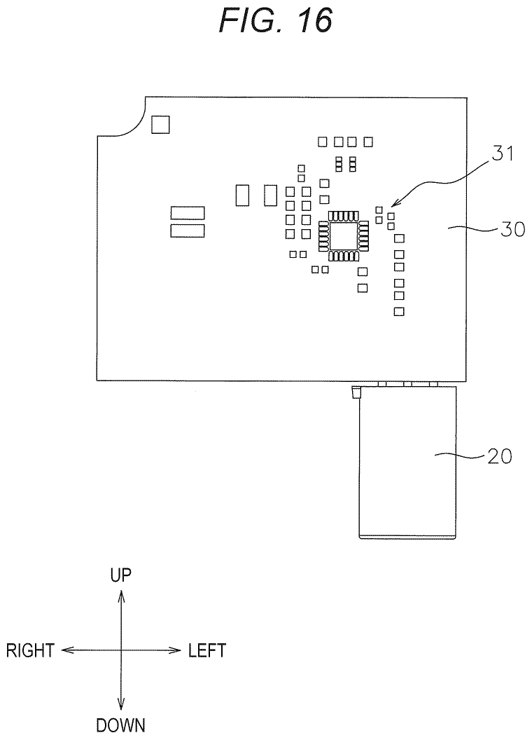

The substrate 30 includes an electric circuit 31 (see FIG. 16). The electric circuit 31 is connected to the gas sensor 20. The electric circuit 31 is, for example, an interconnected assembly of electric and electronic components having a function of extracting a detection result of the gas sensor 20 to the outside as an electric signal and a function of supplying electric power to the gas sensor 20. Examples of components (not illustrated) constituting the electric circuit 31 include an integrated circuit, a capacitor, and a resistor. The gas sensor 20 is connected to, for example, a metal wiring (not illustrated) constituting the electric circuit 31.

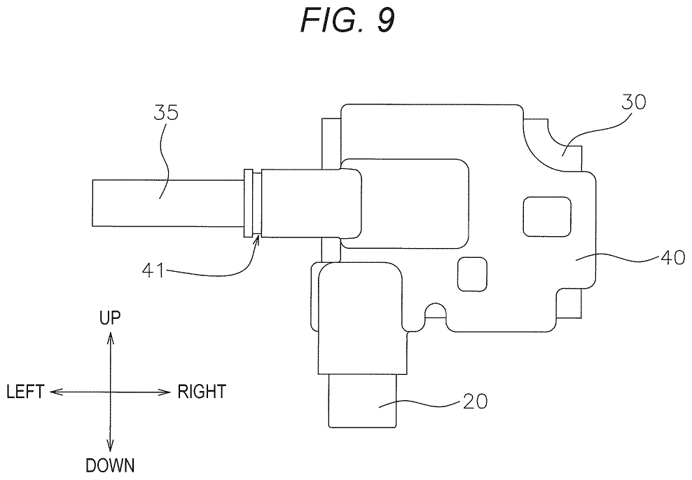

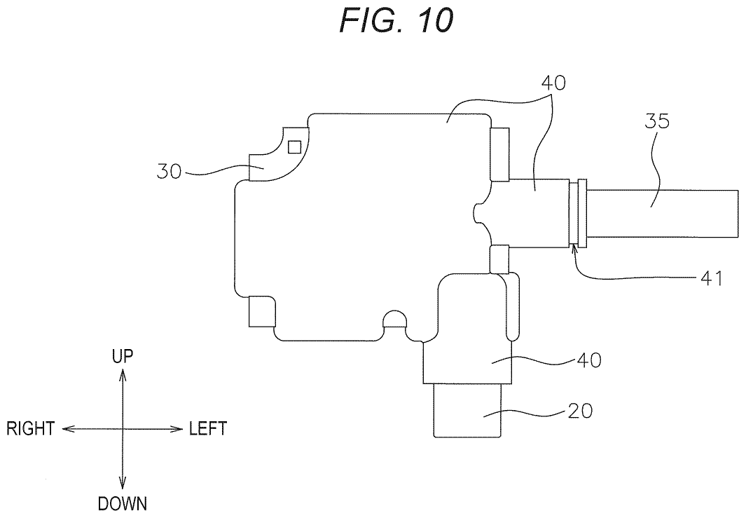

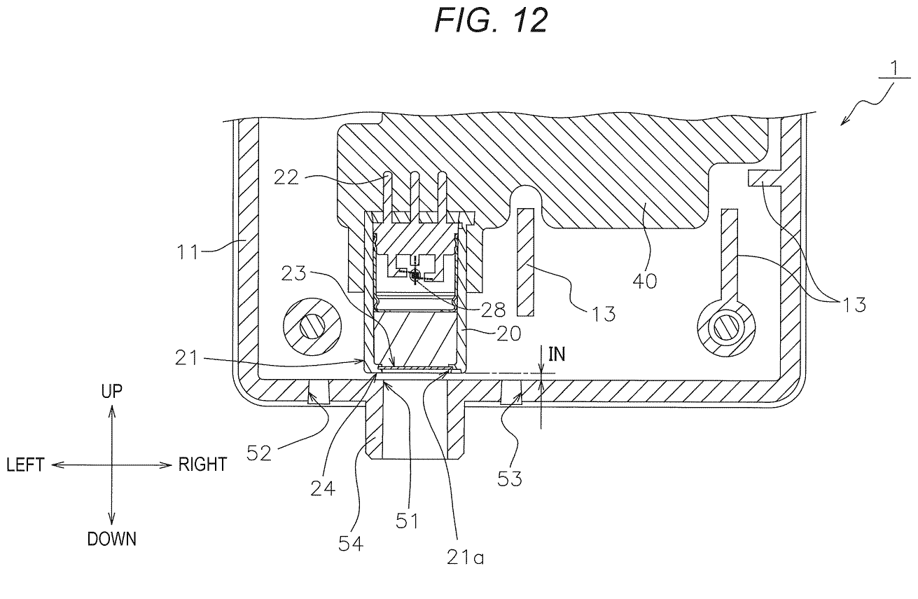

As illustrated in FIGS. 9 and 10, the waterproof molded body 40 is molded so as to cover a portion of the substrate 30 and a portion of the gas sensor 20. The portions covered with the waterproof molded body 40 are waterproofed. The waterproof molded body 40 is molded in a desired shape by, for example, injection molding. A lower portion (a portion including a lower end 24) of the gas sensor 20 is exposed without being covered with the waterproof molded body 40. At least the electric circuit 31 is included in a portion of the substrate 30 covered with the waterproof molded body 40. By covering a portion of the substrate 30 and a portion of the gas sensor 20, the waterproof molded body 40 prevents water from entering the electric circuit 31. The prevention of entry of water into the electric circuit 31 also includes prevention of entry of water into a connection terminal 22 (see FIG. 12) of the gas sensor 20 and a connection portion (not illustrated) of the electric circuit 31. The connection terminal 22 is connected to the connection portion of the electric circuit 31. The connection portion of the electric circuit 31 is, for example, a portion of a metal wiring for constituting the electric circuit 31. The waterproof molded body 40 is formed with a material having insulating property and impermeable to water. The waterproof molded body 40 is, for example, a thermoplastic adhesive, a resin, or an elastomer. The waterproof molded body is preferably formed with a material that can be injection-molded. By inserting and injection molding the substrate 30 and the gas sensor 20, the waterproof molded body 40 can be formed at desired positions of the substrate 30 and the gas sensor 20 in a short period of time. The connection terminal 22 of the gas sensor 20 is a terminal for supplying electric power to a gas-sensitive element 28 (see FIG. 12). The gas-sensitive element 28 is, for example, an element constituting a semiconductor gas sensor.

Examples of the thermoplastic adhesive include a hot melt adhesive. The waterproof molded body 40 is, for example, a hot melt mold molded by hot melt molding using a hot melt adhesive. Examples of the hot melt adhesive include a polyamide-based hot melt adhesive, a polyolefin-based hot melt adhesive, and a polyurethane-based hot melt adhesive. The conditions for injection molding of the hot melt adhesive are preferably selected from low temperature and low pressure in which an injection pressure is 0.2 to 6 MPa and an injection temperature is equal to or higher than 180 °C and equal to or lower than 240 °C. Examples of the elastomer include silicone, urethane rubber, and fluororubber. Examples of the resin include a thermoplastic resin, a thermosetting resin, and a photocurable resin. Examples of the thermoplastic resin include polyethylene, acrylic resin, polypropylene, and polycarbonate. Examples of the thermosetting resin include an epoxy resin, a phenol resin, and a polyurethane resin. Examples of the photocurable resin include an acrylic photocurable resin.

As illustrated in FIG. 7, the container 10 houses the gas sensor 20 and the substrate 30 in an internal space IS. The gas sensor 20 is housed in the internal space IS in the container 10, and thus, it is necessary to introduce the gas to be detected into the internal space IS in the container 10 in order for the gas sensor 20 to detect the gas. Thus, in order to introduce gas heavier than air into the internal space IS, as illustrated in FIG. 2, a gas inlet 51 opened downward is provided in the lower wall 111 of the container 10.

In order to introduce the gas from the gas inlet 51, it is necessary to take out substantially the same amount of air as the gas to be introduced from the internal space IS to the outside of the container 10. If the gas and the air are replaced only by the gas inlet 51, the introduction of the gas is delayed, which causes delay in gas detection. In order to smoothly replace the gas and the air, drain ports 52, 53 functioning as air vent holes for introducing the gas are provided in the lower wall 111 of the container 10. Of course, the drain ports 52, 53 have a function of draining water from the internal space IS of the container 10 to the outside of the container 10. By providing the drain ports 52, 53 in the lower wall 111, drainage can be performed more efficiently and air can be removed more efficiently than in a case where the drain ports are provided in other walls. Here, a case where two drain ports 52, 53 are provided will be described, but one or three or more drain ports may be provided.

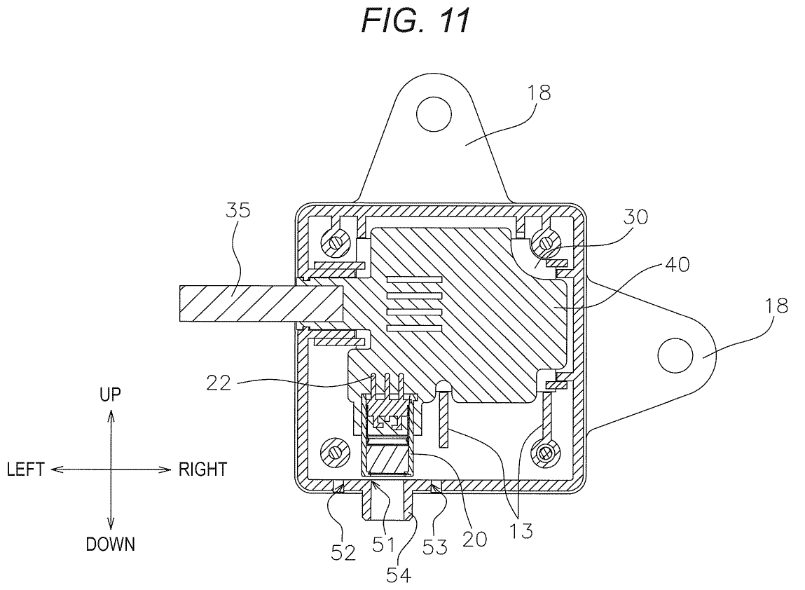

As illustrated in FIGS. 11 and 12, the substrate 30 is fixed to the container 10 so that an opening 21a of the gas sensor 20 faces the gas inlet 51. Thus, the gas to be detected heavier than air that has entered the internal space IS from the gas inlet 51 quickly enters the opening 21a of the gas sensor 20 and is detected by the gas sensor 20. FIGS. 11 and 12 illustrate cross sections of the gas sensor module 1 cut along planes extending vertically and horizontally.

### (2) Detailed Configuration

### (2-1) Positional Relationship Between Gas Sensor 20 and Lower Wall 111

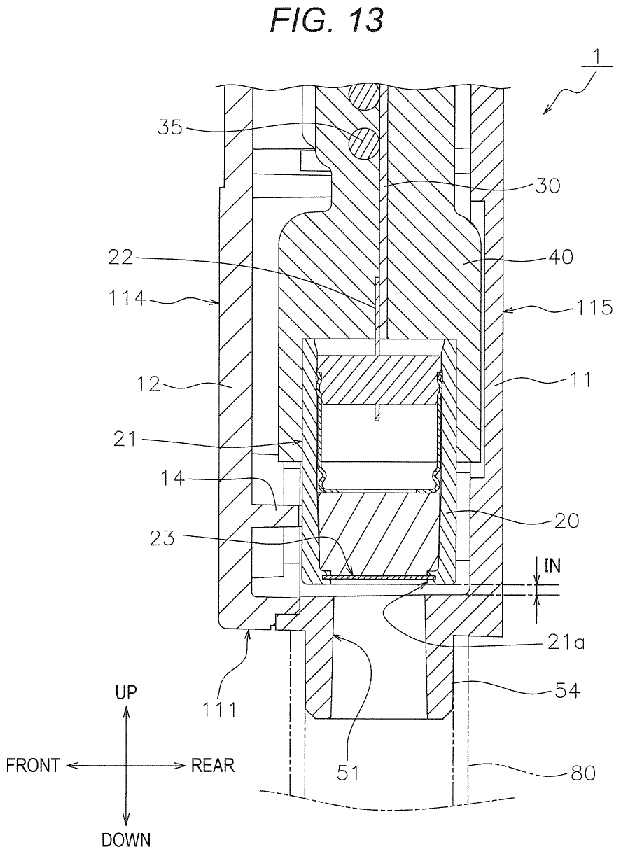

The gas sensor 20 incorporates the gas-sensitive element 28 in a cap 21. The gas sensor 20 detects gas with the gas-sensitive element 28. The lower wall 111 of the container 10 is provided with the gas inlet 51. FIGS. 12 and 13 are enlarged views of a periphery of the gas inlet 51. FIG. 12 is an enlarged cross-sectional view of the periphery of the gas inlet of FIG. 11. FIG. 13 is an enlarged cross-sectional view of the periphery of the gas inlet 51, obtained by cutting the gas sensor module 1 along a plane extending vertically and longitudinally. The container 10 is configured to support the substrate 30 with a gap IN of 0.5 mm or more provided between the lower end 24 of the gas sensor 20 and the lower wall 111. By forming the gap IN of 0.5 mm or more between the lower end 24 of the gas sensor 20 and the lower wall 111, in a case where water droplets are generated on the lower wall 111, the water droplets can be suppressed from moving to the gas sensor 20.

The opening 21a of the gas sensor 20 is disposed to face the gas inlet 51, and the opening 21a has a size substantially matching the gas inlet 51. Here, “substantially” means that there is a difference in a range of manufacturing errors. Reducing a size of one of the opening 21a and the gas inlet 51 leads to reduction in introduction of the gas to be detected. The opening 21a and the gas inlet 51 have a circular shape, and are disposed so that center points substantially coincide with each other when viewed in a vertical direction. Reduction in an overlapping area of the opening 21a and the gas inlet 51 leads to reduction in introduction of the gas to be detected.

If the size of the opening 21a is made to substantially match the size of the gas inlet 51 and an overlapping region of the opening 21a and the gas inlet 51 is maximized, for example, a risk is increased that a spray of minute water droplets flying from the bottom toward the top from the gas inlet 51 toward the opening 21a directly jumps into the gas sensor 20 from the opening 21a. In order to suppress entry of minute water droplets into the gas sensor 20, a net 23 is provided in the opening 21a in this embodiment. The net 23 covers the entire opening 21a. The net 23 is formed with a metal, for example. A mesh size of the net 23 is, for example, 50 mesh to 200 mesh. However, a situation where many water droplets adhere to the net 23 is not preferable because the net 23 is covered with a water film. In a case where the gas sensor module 1 is installed at a location where such a situation is likely to occur, it is preferable to increase the gap IN between the gas sensor 20 and the lower wall 111 to move the gas sensor 20 away from the lower wall 111, so that water droplets are less likely to reach the net 23. However, the net 23 does not have to be provided. In addition, instead of the net 23, for example, an air-permeable member such as a nonwoven fabric, a woven fabric, or a sponge may be provided.

### (2-2) Fixing of Substrate 30 in Container 10

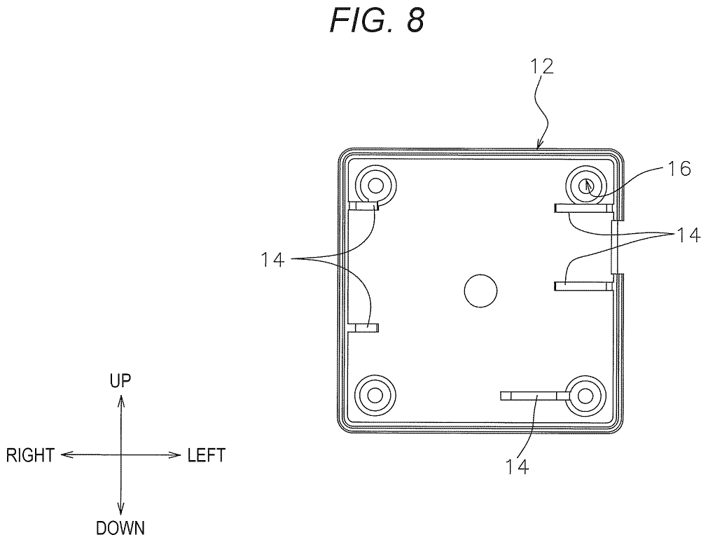

The container 10 includes ribs 13, 14 protruding into the internal space IS in FIGS. 8, 12, and 13. The substrate 30 preferably has higher rigidity than the waterproof molded body 40. The substrate 30 is, for example, a rigid substrate or a ceramic substrate. The rigid substrate is, for example, a substrate obtained by impregnating a glass cloth with a thermosetting resin. Examples of the thermosetting resin to be impregnated include an epoxy resin, an imide resin, a bismaleimide triazine (BT) resin, and a polyphenylene ether (PPE) resin.

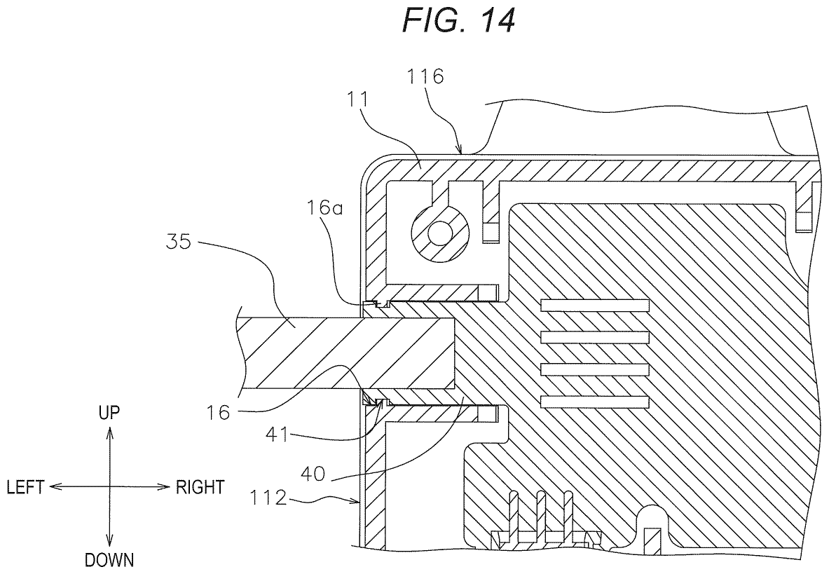

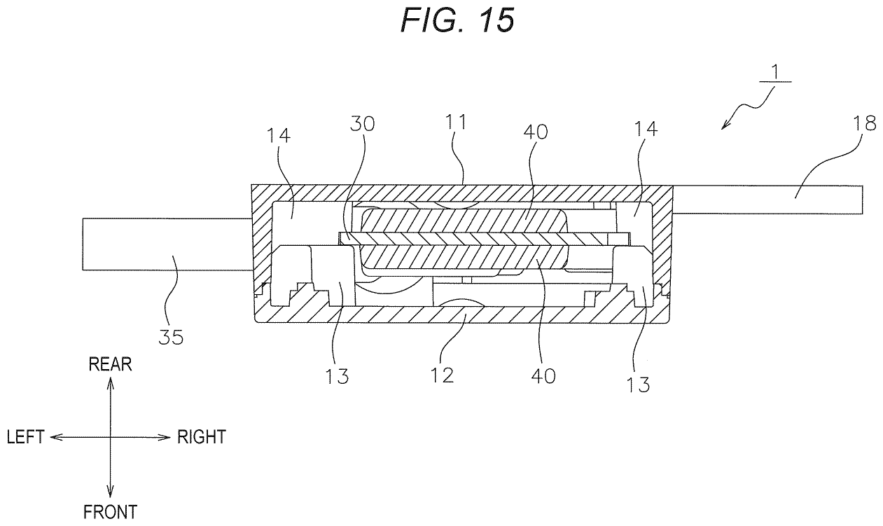

For example, in a case where a hot melt adhesive is selected as the material of the waterproof molded body 40, a rigid substrate or a ceramic substrate is used so that the rigidity of the substrate 30 is higher than the rigidity of the waterproof molded body 40. Assuming that the ribs 13, 14 are made to abut on the waterproof molded body 40 formed with a hot melt adhesive, it is difficult to maintain the gap IN because the hot melt adhesive is deformed. Thus, the ribs 13, 14 do not abut on the waterproof molded body 40, but abut on the substrate 30 to support the substrate 30. In other words, a portion of the substrate 30 exposed from the waterproof molded body 40 is supported by the ribs 13, 14, and the substrate 30 is fixed by being sandwiched between the two types of the rib 13 and the rib 14 (see FIG. 15). The rib 13 is provided in the housing portion 11, and the rib 14 is provided in the lid portion 12. By attaching the lid portion 12 to the container 10 with the screws 90, the substrate 30 is sandwiched between the ribs 13, 14 and fixed to the container 10. The container 10 is mainly in contact with the substrate 30 by the ribs 13, 14 except for being in contact with the cable 35 at the cable outlet 16 (see FIG. 14). The substrate 30 is supported by the ribs 13, 14 to reduce a contact portion of the container 10 with respect to the gas sensor 20, the substrate 30, and the waterproof molded body 40.

### (2-3) Shape of Periphery of Gas Inlet 51

As illustrated in FIG. 1, the container 10 includes a protrusion 54 around the gas inlet 51. The protrusion 54 protrudes downward from the lower wall 111. As illustrated in FIG. 2, a shape of the protrusion 54 is a cylinder. An internal cavity of the cylindrical protrusion 54 serves as the gas inlet 51. Inspection equipment 80 (see FIG. 13) is attached to the protrusion 54. The inspection equipment 80 includes, for example, a hose that is fitted into the protrusion 54 and sends gas for inspection. If the protrusion 54 is not provided and the lower wall 111 has a flat plate shape, it is difficult to attach the inspection equipment 80. However, as a result of the protrusion 54 being provided, it is easy to attach the inspection equipment 80. A shape of the protrusion 54 does not have to be a cylinder, and may be, for example, a square tube. However, the shape of the protrusion 54 is preferably a cylinder to which the hose is easily attached. As a result of the protrusion 54 being provided as described above, a range of water droplets bouncing from the bottom to the top is narrowed as compared with a case where the protrusion 54 is not provided.

### (2-4) Arrangement Position and Shape of Drain Ports 52,53

As illustrated in FIG. 2, on the lower wall 111 of the container 10, the gas inlet 51 is disposed between the two drain ports 52, 53. As illustrated in FIG. 12, the drain ports 52, 53 are disposed on the left and right sides of the gas sensor 20, respectively.

The drain ports 52, 53 are a first elongated hole and a second elongated hole extending long in a front-rear direction. A width of the first elongated hole and the second elongated hole is preferably 2 mm or more. If the width is smaller than 2 mm, it is difficult to drain water from the drain ports 52, 53. A length of the first elongated hole and the second elongated hole is preferably equal to or longer than the length of the gas inlet 51 in the front-rear direction. In this case, water droplets flowing from the left of the drain port 52 toward the gas sensor 20 or water droplets flowing from the right of the drain port 53 toward the gas sensor 20 are easily drained to the outside of the container 10 before reaching the gas sensor 20. It is difficult for the lower wall 111 of the fixed container 10 to be horizontal, and thus, it is preferable to provide the drain ports 52, 53 not on one side but on both sides of the gas inlet 51. Note that a shape of the drain ports 52, 53 does not have to be an elongated hole, and may be, for example, a circle, an ellipse, or a rectangle. However, a distance of the narrowest portion of the drain ports 52, 53 is preferably 2 mm or more for drainage.

### (2-5) Structure of Periphery of Cable Outlet 55

The gas sensor module 1 includes a cable 35. The cable 35 extending from the container 10 is illustrated in FIG. 1. As illustrated in FIGS. 9 and 10, the gas sensor 20, the substrate 30, the waterproof molded body 40, and the cable 35 are integrated into an assembly. The cable 35 is a member for electrically connecting the electric circuit 31 housed in the container 10 and equipment outside the container 10. The cable 35 includes a plurality of covered electric wires (not illustrated). The cable 35 extends to the outside of the container 10 through a cable outlet 55 provided on the left side wall 112. The cable 35 is connected to, for example, the connection portion (not illustrated) of the electric circuit 31.

The container 10 includes a cable outlet 16 for taking out the cable 35 from the substrate 30 to the outside of the container 10. The waterproof molded body 40 covers a portion of the cable 35 and fills a gap between the cable outlet 16 and the cable 35 to seal the cable outlet 16. For sealing, a portion where the waterproof molded body 40 covers the cable 35 extends to the cable outlet 16. As illustrated in FIG. 12, the cable outlet 16 extends in a cylindrical shape toward the internal space IS. The cable outlet 16 is provided with a bank portion 16a protruding inward over the entire circumference of the cable outlet 16. The waterproof molded body 40 is formed with a groove portion 41 into which the bank portion 16a is to be fitted. The waterproof molded body 40 is, for example, a hot melt mold hot-melt-molded in the shape of the cable outlet 16.

### (3) Modifications

### (3-1) Modification A

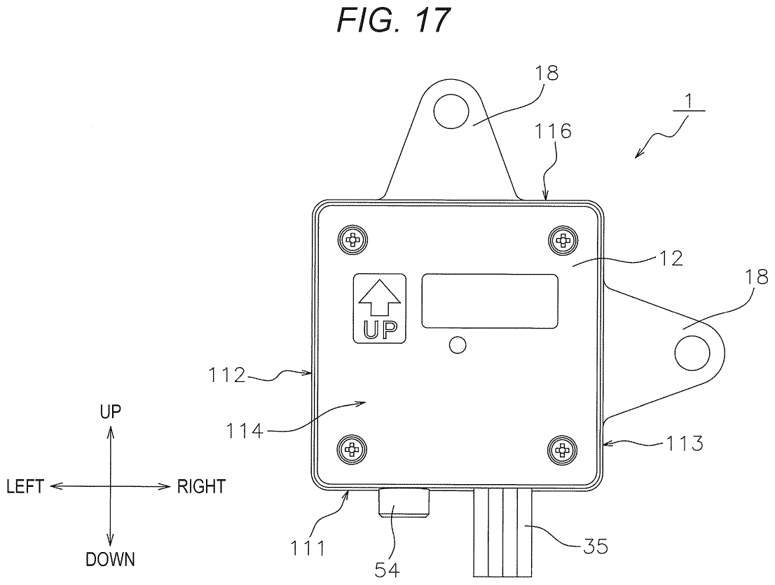

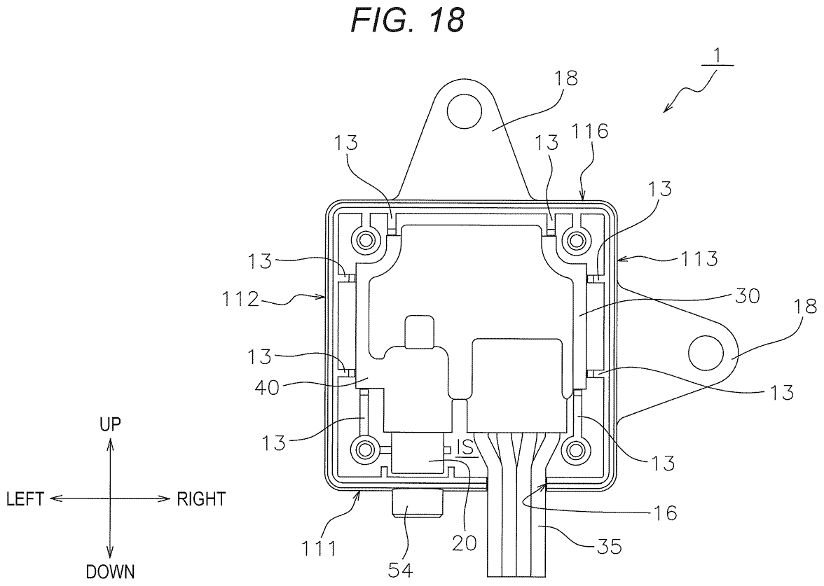

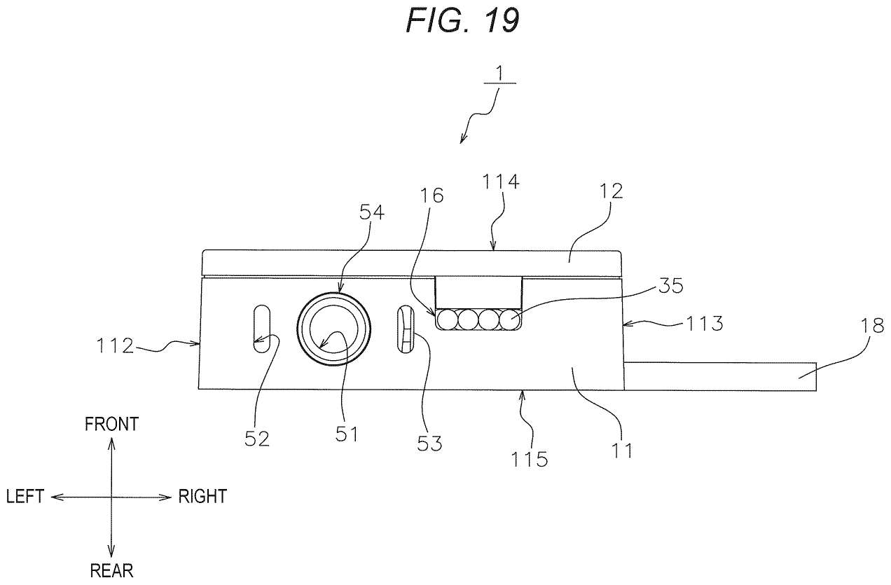

In the above embodiment, a case where the cable 35 extends from the left side wall 112 of the container 10 to the outside of the container 10 has been described. However, the arrangement position of the cable outlet 16 of the cable 35 is not limited to the left side wall 112. The cable outlet 16 may be provided on the right side wall 113, for example. In addition, as illustrated in FIGS. 17, 18, and 19, the cable outlet 16 may be provided on the lower wall 111, and the cable 35 may extend downward. By providing the cable outlet 16 on the left side wall 112, the right side wall 113, or the lower wall 111, water is less likely to enter the internal space IS of the container 10 from the outside of the container 10 as compared with a case where the cable outlet 16 is provided on the upper wall 116. Note that while the cable 35 illustrated in FIG. 19 is not covered with the waterproof molded body 40 up to the portion of the cable outlet 16, the portion of the cable outlet 16 may be covered with the waterproof molded body 40 as in the embodiment.

### (3-2) Modification B

In the above embodiment, a case where the substrate 30 is fixed to the container 10 by the ribs 13, 14 has been described as a method for fixing the substrate 30, but a method other than fastening by the ribs 13, 14 may be used. For example, the substrate 30 may be fixed to the container 10 using a screw or an adhesive. Even in a case where the substrate 30 is fixed to the container 10 using a screw or an adhesive, it is preferable that the assembly of the gas sensor 20, the substrate 30, the waterproof molded body 40, and the cable 35 is fastened to the inner surface of the container 10 so as not to be in contact with the inner surface as much as possible. In a case where an adhesive is used, it is preferable to use an adhesive having a composition in which gas is hardly generated from the adhesive after curing, such as an epoxy-based adhesive.

### (3-3) Modification C

In the above embodiment, a case where communication between the electric circuit 31 of the substrate 30 and the outside is performed by the cable 35 has been described. However, without using the cable 35, for example, the electric circuit 31 and equipment outside the container 10 may perform communication in a wireless manner. For example, communication of electric signals and charging may be performed by electromagnetic waves.

### (4) Features

(4-1)

The gas sensor module 1 includes the gas sensor 20, the substrate 30, the waterproof molded body 40, and the container 10. The gas sensor 20 is a gas sensor that detects gas heavier than air. The substrate 30 includes the electric circuit 31 connected to the gas sensor 20. The waterproof molded body 40 covers a portion of the substrate 30 and a portion of the gas sensor 20 to waterproof the covered portions. A portion of the substrate 30 covered with the waterproof molded body 40 includes the electric circuit 31. In particular, a portion of the substrate 30 includes a connection portion for connecting to the gas sensor 20 and the cable 35. The container 10 houses the gas sensor 20 and the substrate 30 in the internal space IS. A portion of the gas sensor 20 covered with the waterproof molded body 40 includes at least the connection terminal 22 for connecting to the connection portion of the electric circuit 31.

The container 10 includes the gas inlet 51 and the drain ports 52, 53 on the lower wall 111. For example, in a case where gas heavier than air leaks inside equipment to which the gas sensor module 1 is attached, the gas accumulates at a lower portion of the equipment, and a situation in which a boundary between the air and the gas gradually moves upward is likely to occur. Thus, if the gas inlet 51 is opened downward to introduce gas heavier than air into the internal space IS, the leaked gas is easily detected. The drain ports 52, 53 are openings for draining water that has entered the internal space IS. These drain ports 52, 53 also function as air vent holes for introducing gas. As illustrated in FIG. 12, the gas-sensitive element 28 is located in the cap 21 and above the opening 21a. In order to introduce the gas into the internal space IS, part of the air in the internal space IS needs to go out of the container 10. The drain ports 52, 53 can function as a discharge port for air to be discharged from the internal space IS to the outside of the container 10 in exchange for gas to assist replacement of the gas and the air. As a result of the drain ports 52, 53 being responsible for discharging air and discharging water, the number of holes formed in the lower wall 111 can be reduced to suppress water from entering the internal space IS.

The substrate 30 is fixed to the container 10 so that the opening 21a of the gas sensor 20 faces the gas inlet 51. By fixing the substrate 30 in this manner, the gas can be easily guided from the outside of the container 10 to the gas-sensitive element 28 in the gas sensor 20 through the gas inlet 51 and the opening 21a.

The container 10 having the above-described configuration can prevent water from being splashed on the gas sensor 20 and the substrate 30 while allowing gas outside the container 10 to be guided to the gas sensor 20. Furthermore, the waterproof molded body 40 can prevent water from entering the electric circuit 31.

In a case where the waterproof molded body 40 is molded by injection molding, it is easy to manufacture an assembly including the gas sensor 20, the substrate 30, the waterproof molded body 40, and the cable 35.

(4-2)

In the gas sensor module 1, the substrate 30 is supported with the gap IN of 0.5 mm or more between the lower end 24 of the gas sensor 20 and the lower wall 111. In the gas sensor module 1, the gap IN of 0.5 mm or more exists between the lower end 24 of the gas sensor 20 and the lower wall 111, so that water droplets generated on the lower wall 111 of the container 10 hardly enter the gas sensor 20.

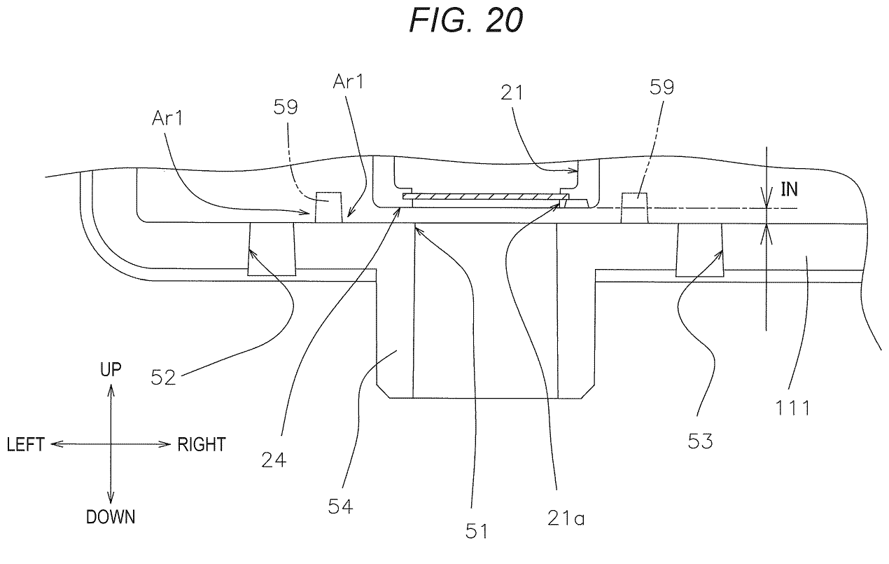

In addition, it is conceivable to form the bank portion 59 protruding upward and extending along the front-rear direction as illustrated in FIG. 20 on the upper surface of the lower wall 111 so that water droplets on the lower wall 111 do not flow toward the gas sensor 20. However, it has been confirmed by experiments that if such a bank portion 59 is formed, dust is accumulated in a neighboring area Arl of a boundary between an upper surface of the lower wall 111 and a side surface of the bank portion 59. Dust also adversely affects the gas sensor 20, and thus, it is preferable not to form a portion protruding upward from the upper surface of the lower wall 111 like the bank portion 59. For example, it is possible to cope with water droplets on the upper surface of the lower wall 111 by adjusting a size of the gap IN between the lower end 24 of the gas sensor 20 and the lower wall 111. In other words, the upper surface of the lower wall 111 is preferably flat. Alternatively, a smooth slope may be provided on the upper surface of the lower wall 111 to encourage drainage.

(4-3)

In the gas sensor module 1, a portion of the substrate 30 exposed from the waterproof molded body 40 is supported and fixed by ribs 13, 14. As a result of the substrate 30 being supported by the ribs 13, 14, it is possible to reduce the number of places where the gas sensor 20, the substrate 30, and the waterproof molded body 40 are in contact with the inner surface of the container, so that it is possible to suppress water from being transferred from the container 10 to the gas sensor 20, the substrate 30, and the waterproof molded body 40.

In addition, in a case where the waterproof molded body 40 is formed with a hot melt mold, and the hot melt mold portion is supported, positioning of the gas sensor 20 becomes difficult because the waterproof molded body 40 is soft. By directly supporting the substrate 30 exposed from the waterproof molded body 40 by the ribs 13, 14, the substrate 30 having higher rigidity than the waterproof molded body 40 is supported, so that the gas sensor 20 can be easily positioned, and particularly, the gap IN of 0.5 mm or more between the lower end 24 of the gas sensor 20 and the lower wall 111 can be easily secured. Further, use of the hot melt mold as the waterproof molded body 40 improves waterproof property.

(4-4)

For example, a hose of inspection equipment can be attached to the protrusion 54 around the gas inlet 51 in the gas sensor module 1. If the protrusion 54 is provided, the inspection equipment can be easily attached as compared with a case where the protrusion 54 is not provided and the lower surface of the lower wall 111 is flat. In addition, in a case where water droplets are splashed from obliquely below the gas inlet 51 toward the gas inlet 51, some of the water droplets are blocked by the protrusion 54, so that entry into the gas inlet 51 can be reduced.

(4-5)

In the gas sensor module 1, the drain port 52 illustrated in FIG. 2 is a first elongated hole, and the drain port 53 is a second elongated hole. Further, the drain ports 52, 53 are provided on both sides of the gas inlet 51. Capability of draining water droplets flowing from the left side of the drain port 52 and the right side of the drain port 53 when viewed from the front, toward the gas sensor 20 including the opening 21a facing the drain port 52 is improved. In addition, it is possible to increase the opening area while making it difficult for water droplets from the outside to enter the internal space IS. Thus, the drain ports 52, 53 can have high drainage capacity and high functionality as the air vent hole.

(4-6)

The gas sensor module 1 includes the cable 35 connected to the electric circuit 31. The waterproof molded body 40 covers a portion of the cable 35 and fills a gap between the cable outlet 16 and the cable 35 to seal the cable outlet 16. The cable outlet 16 is sealed by the waterproof molded body 40, so that it is possible to prevent water from entering the internal space IS of the container 10 along the cable 35.

In particular, by fitting the bank portion 16a of the cable outlet 16 into the groove portion 41 of the waterproof molded body 40, sealability of the waterproof molded body 40 is improved.

(4-7)

In the method for manufacturing the gas sensor module 1, for example, in the first step, the waterproof molded body 40 is formed by hot melt molding so that a portion of the gas sensor 20 that detects gas heavier than air and a portion of the substrate 30 including the electric circuit 31 in the substrate 30 including the electric circuit 31 connected to the gas sensor 20 are covered with the waterproof molded body 40. In the second step, the substrate 30 is made to abut on the container 10 and the substrate 30 is fixed to the container 10 so that the opening 21a of the gas sensor 20 faces the gas inlet 51 of the container 10 opened downward to introduce gas heavier than air into the internal space IS of the container 10, and the gap IN is formed between the lower end 24 of the gas sensor 20 and the lower wall 111 of the container 10.

In the embodiment, the substrate 30 abuts on the ribs 13, 14 which are components of the container 10.

If such a method for manufacturing the gas sensor module 1 is used, a portion of the gas sensor 20 and a portion of the substrate 30 can be quickly and accurately covered with the waterproof molded body 40 by hot melt molding. Further, in the method for manufacturing the gas sensor module 1, the substrate 30 is made to abut on the container 10, and thus, the substrate 30 can be easily positioned as compared with a case where the waterproof molded body 40 formed by hot melt molding is made to abut on the container 10. This results in facilitating positioning of the gas sensor 20 inside the container 10 and makes it possible to easily manufacture the gas sensor module 1 with improved waterproof property.

1. A gas sensor module comprising:
a gas sensor configured to detect gas heavier than air;
a substrate including an electric circuit connected to the gas sensor;
a waterproof molded body covering a portion of the substrate including the electric circuit and a portion of the gas sensor to waterproof the covered portions; and
a container housing the gas sensor and the substrate in an internal space, the container including a gas inlet opened downward to introduce the gas heavier than air into the internal space, and a drain port that functions as an air vent hole for introducing the gas and drains water entering the internal space,
the substrate being fixed to the container so that an opening of the gas sensor faces the gas inlet.

2. The gas sensor module according to claim 1, wherein
the container has a lower wall provided with the gas inlet, and
the container supports the substrate with a gap of 0.5 mm or more between a lower end of the gas sensor and the lower wall.

3. The gas sensor module according to claim 1, wherein
the container includes a rib protruding into the internal space,
the waterproof molded body is a hot melt mold,
the substrate has higher rigidity than the waterproof molded body, and
a portion of the substrate exposed from the waterproof molded body is supported and fixed by the rib.

4. The gas sensor module according to claim 1, wherein
the container includes a protrusion around the gas inlet for attaching inspection equipment.

5. The gas sensor module according to claim 1, wherein
the container includes, as the drain port, a first elongated hole and a second elongated hole provided on both sides of the gas inlet.

6. The gas sensor module according to claim 1, further comprising
a cable connected to the electric circuit,
the container including a cable outlet for taking out the cable from the substrate to the outside of the container, and
the waterproof molded body covering a portion of the cable and filling a gap between the cable outlet and the cable to seal the cable outlet.

7. A method for manufacturing a gas sensor module, the method comprising:
forming a waterproof molded body by hot melt molding so that a portion of a gas sensor that is configured to detect gas heavier than air and a portion of a substrate including an electric circuit connected to the gas sensor in the substrate that includes the electric circuit connected to the gas sensor are covered with the waterproof molded body; and
making the substrate abut on a container to fix the substrate to the container so that an opening of the gas sensor faces a gas inlet of the container opened downward for introducing the gas heavier than air into an internal space of the container, and a gap is formed between a lower end of the gas sensor and a lower wall of the container.

8. The gas sensor module according to claim 2, wherein
the container includes a rib protruding into the internal space,
the waterproof molded body is a hot melt mold,
the substrate has higher rigidity than the waterproof molded body, and
a portion of the substrate exposed from the waterproof molded body is supported and fixed by the rib.

9. The gas sensor module according to claim 2, wherein
the container includes a protrusion around the gas inlet for attaching inspection equipment.

10. The gas sensor module according to claim 2, wherein
the container includes, as the drain port, a first elongated hole and a second elongated hole provided on both sides of the gas inlet.

11. The gas sensor module according to claim 2, further comprising
a cable connected to the electric circuit,
the container including a cable outlet for taking out the cable from the substrate to the outside of the container, and
the waterproof molded body covering a portion of the cable and filling a gap between the cable outlet and the cable to seal the cable outlet.
